(19) United States
(12) Patent Application Publication
(10) Pub. No.: US 2003/0163205 A1
(43) Pub. Date: Aug. 28, 2003

(54) Arthroplasty-prothesis cement restrictor

(76) Inventor: Kevin Jon Lawson, Redding, CA (US)

Correspondence Address: Robert Charles Hill, 235 Montgomery Street #821, San Francisco, CA, 94104, US

(21) Appl. No.: 10/084,108

(22) Filed: Feb. 27, 2002

(51) Int. Cl. A61F 2/28; A61F 2/36

(52) U.S. Cl. 623/23.48; 606/95

(57) ABSTRACT

An arthroplasty prosthesis cement restrictor comprises a cylinderlike body having a hexagonal lateral outside cross section. A set of corresponding longitudinal external ridges are disposed along the points of the hexagonal lateral cross section. A spiraling machine-thread is formed in the set of corresponding longitudinal ridges and provides for a threaded positive lock of the cylinderlike body into the walls of a long-bone intramedullary canal. A capped end is disposed at one end of the cylinderlike body and provides for the stoppage of cement injected during arthroplasty into the more distal parts of the intramedullary canal. A number of corresponding longitudinal fenestrations are disposed between the set of corresponding of longitudinal ridges which can be flooded with cement to allow cement contact with the adjacent bone.

### BACKGROUND OF THE INVENTION

1. Field of the Invention

The present invention relates to surgical methods and devices for artificial joint replacement, and more particularly to plugs and restrictors that fit into the top of the hollow part of long bones to contain arthroplasty cement.

2. Description of Related Art

Total hip replacements have allowed patients with degenerated and dysfunctional hip joints to return to relatively active normal lives. The typical total hip arthroplasty typically involves cementing a cobalt-chromium alloy or titanium-alloy ball stem with methyl-methacrylate to the opened top of the affected femur. The degenerated joint is removed during a surgical procedure, and the ball stem is cemented within a top opening in the hollow bone. The bone is shaped inside and top to receive and hold the ball stem with special cement.

Such stem is only a few centimeters long, and the cement needs to be blocked from going too far into the hollow interior of the femur. Blocking the cement flow helps conserve the amount of material needed to be injected. As far back as 1986, total hip replacement procedures included plugging the medullary canal with a bolus of bone cement, and then retrograde filling with doughy simplex-P methyl-methacrylate from a cement gun.

Ronald M. Carn describes a bone plug for this purpose in U.S. Pat. No. 5,662,657, issued Sep. 2, 1997. Such bone plug is inserted into the top end of the exposed medullary canal in the femur during total hip replacement. Such is said to resist the pressurized cement being introduced to bed the stem. Pressurizing the cement helps with more complete bonding. The bone plug has polyethylene body and fins that seal inside the femur. A push-in type insertion tool for this is also described. This type of plug relies on a tight fit and friction for the plug to stay put. There is no positive locking of the plug to the inside of the bone.

A later patent by Ronald M. Carn, U.S. Pat. No. 5,782,917, issued Jul. 21, 1998, describes a bone plug with a more positive lock. (See also, Ronald M. Carn, U.S. Pat. No. 5,861,043, issued Jan. 19, 1999.) A solid polyethylene corkscrew plug is threaded-in with a twist-type driving tool. Large, coarse threads bite into the interior walls of the medullary canal. Such plug does not allow cement to get between it and the adjacent bone.

### SUMMARY OF THE INVENTION

An object of the present invention is to provide a plug to prevent cement from invading too far into the intramedullary canal during arthroplasty.

Another object of the present invention is to provide a cement restrictor that positively locks into the bone walls of the intramedullary canal during arthroplasty.

A further object of the present invention is to provide a cement restrictor that allows cement to invade it for cement bonding with the adjacent bone.

A still further object of the present invention is to provide a cement restrictor that can be readily adjusted to sit at various positions within the intramedullary canal during arthroplasty.

Briefly, an arthroplasty prosthesis cement restrictor embodiment of the present invention comprises a bullet-shaped body with a hexagonal frontal cross section. A set of corresponding longitudinal ridges are disposed along the points of the hexagonal lateral cross section. A spiraling machine-thread is formed in the set of corresponding longitudinal ridges and provides for a threaded positive lock of the cylinderlike body into the walls of a long-bone intramedullary canal. A capped end is disposed at one end of the cylinderlike body and provides for the stoppage of cement injected during arthroplasty into the more distal parts of the intramedullary canal. A number of corresponding longitudinal fenestrations are disposed between the set of corresponding longitudinal ridges which can be flooded with cement to allow cement contact with the adjacent bone.

An advantage of the present invention is that a cement restrictor is provided for arthroplasty.

Another advantage of the present invention is that a cement restrictor is provided that will not migrate under cement pressure.

A further advantage of the present invention is that a cement restrictor is provided that can be readily depth-adjusted.

The above and still further objects, features, and advantages of the present invention will become apparent upon consideration of the following detailed description of specific embodiments thereof, especially when taken in conjunction with the accompanying drawings.

### BRIEF DESCRIPTION OF THE DRAWINGS

FIG. 1 is a cross sectional diagram representing an arthroplasty embodiment of the present invention after being done by a surgeon;

FIG. 2A is a near or top end view of a cement restrictor embodiment of the present invention similar to that shown in FIG. 1; and

FIG. 2B is a side view of the cement restrictor of FIG. 2A and is similar to that shown in FIG. 1.

### DETAILED DESCRIPTION OF THE INVENTION

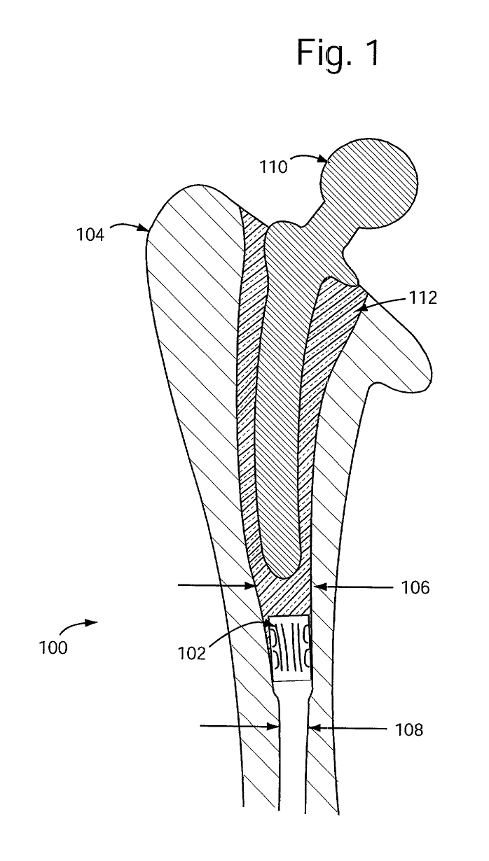

FIG. 1 represents an arthroplasty embodiment of the present invention, referred to herein by the general reference numeral 100. The arthroplasty 100 comprises placing an ultra-high molecular weight polyethylene cement restrictor 102 inside the intramedullary canal of a longbone 104. For example, inside the femur for a total hip replacement, or the tibia for a total knee replacement, or humerus for a total shoulder replacement. A reamed-out portion 106 of the intramedullary canal is prepared to accept the cement restrictor 102. The diameters of reamed-out portion 106 and of the cement restrictor 102 are such that any machine threads on the outside of the cement restrictor 102 will bite into the bone walls and lock-in securely. For example, a 0.5 millimeter difference in the diameters is expected to give good results.

The cement restrictor 102 is twisted in like a set-screw inside a long sleeve nut, and its depth is adjusted by the surgeon by advancing the threading in more, or backing it out. An Allen wrench or square drive tool can be used for this purpose. A natural portion 108 of the intramedullary canal is shown below the cement restrictor 102.

A cobalt-chromium alloy or titanium-alloy ball stem 110 is cemented into the reamed-out portion 106 with methyl-methacrylate cement 112. At least two millimeters of cement mantle are preferred all around the stem. Other metals for the stem and other types of cement can be used. Very often a plastic cup is mounted to the top of stem 110 to fit into the hip socket.

Complete contact of the cement 112 with the ball stem 110 and the reamed-out portion 106 is important to prevent loosening years later. Therefore, the cement 112 is introduced as a doughy paste from an injection gun under pressure. The cement restrictor 102 must not move from its desired position during this pressurized injection of the cement.

It is critical to the arthroplasty 100 that the construction of the cement restrictor 102 includes a distal cap at the bottom, a hollow internal core, and windows or fenestrations to the sides. The cap prevents entry of the cement into the natural portion 108 of the intramedullary canal. The openings allow the cement 112 to invade the hollow internal core and seep out through the fenestrations to contact the adjacent bone walls above the cap end.

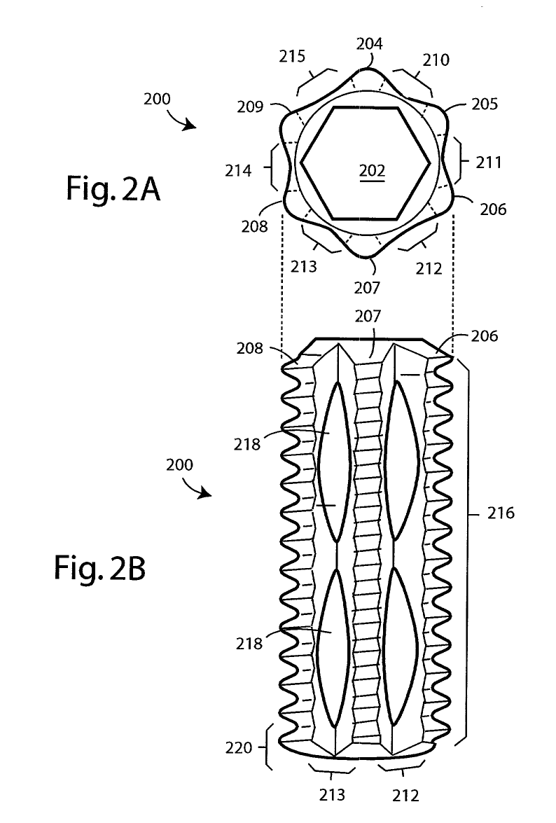

FIGS. 2A and 2B represent a cement restrictor embodiment of the present invention, and is referred to herein by the general reference numeral 200. Such is similar to that used in FIG. 1. The cement restrictor 200 comprises a hollow central core 202 that is open at a near, or top end, and closed at a distal, or bottom end. The internal walls of the hollow central core 202 are preferably shaped to fit and receive a hexagonal Allen wrench or square-drive ratchet. The cement restrictor 200 has a cylinderlike body with a series of longitudinal raised ridges 204-209. Between these are disposed corresponding furrows 210-215. In the embodiment shown in FIGS. 2A and 2B, the external surface of the cement restrictor 200 is generally that of a cylindrical hexagon.

In FIG. 2B, spiraling machine-threads 216 are formed into the tops of ridges 204-209. These can be cut or molded. The furrows have openings 218 to the internal core 202 that allow cement to flood through and contact the bone walls engaged by machine threads 216 in the intramedullary canal. A distal cap 220 prevents any pressurized cement inside hollow core 202 from oozing past. The openings 218 can be one or more per furrow 210-215, and are also referred to as windows or fenestrations.

The cement restrictor 200 comprises a biocompatible material, such as ultra-high molecular weight polyethylene or other polymer. Titanium or carbon-fiber materials could probably also produce acceptable results.

Although particular embodiments of the present invention have been described and illustrated, such is not intended to limit the invention. Modifications and changes will no doubt become apparent to those skilled in the art, and it is intended that the invention only be limited by the scope of the appended claims.

The invention claimed is:

1. An arthroplasty prosthesis cement restrictor, comprising:
a cylinderlike body having approximately hexagonal lateral cross section;
a set of corresponding longitudinal ridges run along the points of said hexagonal lateral cross section;
a spiraling machine-thread formed in the set of corresponding longitudinal ridges and providing for a threaded positive lock of the cylinderlike body into the walls of an intramedullary canal;
a capped end disposed at one end of the cylinderlike body and providing for the stoppage of cement injected during arthroplasty into the more distal parts of said intramedullary canal; and
a plurality of corresponding longitudinal fenestrations disposed between the set of corresponding longitudinal ridges which can be flooded with cement to allow cement contact with an adjacent bone.

2. The arthroplasty prosthesis cement restrictor of claim 1, wherein:
the cylinderlike body comprises a biocompatible polymer material.

3. The arthroplasty prosthesis cement restrictor of claim 1, further comprising:
a hollow, posterior drive section disposed within the central axis of the cylinderlike body and providing for the reception of a twisting-in driving force.

4. The arthroplasty prosthesis cement restrictor of claim 1, further comprising:
a hollow, posterior core disposed within the central axis of the cylinderlike body and opening out to the plurality of corresponding longitudinal fenestrations.

5. The arthroplasty prosthesis cement restrictor of claim 4, further comprising:
a fill of cement flooded into the hollow, posterior core and the plurality of corresponding longitudinal fenestrations for contacting with said adjacent bone wall.
